(19) United States
(12) Patent Application Publication
(10) Pub. No.: US 2008/0300620 A1
(43) Pub. Date: Dec. 4, 2008

(54) EMBOLIC FILTER MADE FROM A COMPOSITE MATERIAL

(75) Inventor: Andrzej J. Chanduszko, Chandler, AZ (US)

Correspondence Address: C.R. Bard, Inc.;Bard Peripheral Vascular, 1625 W. 3rd St, PO Box 1740, Tempe, AZ, 85280-1740, US

(73) Assignee: C.R. BARD, INC., Murray Hill, NJ (US)

(21) Appl. No.: 11/756,107

(22) Filed: May 31, 2007

(51) Int. Cl. A61M 29/00

(52) U.S. Cl. 606/200

(57) ABSTRACT

An embolic filter is disclosed and includes a hub and at least one elongated member extending from the hub. The at least one elongated member includes a core and a jacket circumscribing the core. A ratio of a core diameter to a jacket diameter is at least 0.60.

### FIELD OF THE DISCLOSURE

The present disclosure relates generally to surgical devices. More specifically, the present disclosure relates to embolic filters.

### BACKGROUND

A pulmonary embolism (PE) is a blockage of the pulmonary artery, or a branch of the pulmonary artery, by a blood clot, fat, air, a clump of tumor cells, or other embolus. The most common form of pulmonary embolism is a thromboembolism. A thromboembolism can occur when a venous thrombus, i.e., a blood clot, forms in a patient, becomes dislodged from the formation site, travels to the pulmonary artery, and becomes embolized in the pulmonary artery. When the blood clot becomes embolized within the pulmonary artery and blocks the arterial blood supply to one of the patient's lungs, the patient can suffer symptoms that include difficult breathing, pain during breathing, and circulatory instability. Further, the pulmonary embolism can result in death of the patient.

Commons sources of embolism are proximal leg deep venous thrombosis (DVTs) and pelvic vein thromboses. Any risk factor for DVT can also increase the risk that the venous clot will dislodge and migrate to the lung circulation. One major cause of the development of thrombosis includes alterations in blood flow. Alterations in blood flow can be due to immobilization after surgery, immobilization after injury, and immobilization due to long-distance air travel. Alterations in blood flow can also be due to pregnancy and obesity.

A common treatment to prevent pulmonary embolism includes anticoagulant therapy. For example, heparin, low molecular weight heparins (e.g., enoxaparin and dalteparin), or fondaparinux can be administered initially, while warfarin therapy is commenced. Typically, warfarin therapy can last three to six months. However, if a patient has experienced previous DVTs or PEs, warfarin therapy can last for the remaining life of the patient.

If anticoagulant therapy is contraindicated, ineffective, or a combination thereof, an embolic filter can be implanted within the inferior vena cava of the patient. An embolic filter, i.e., an inferior vena cava filter, is a vascular filter that can be implanted within the inferior vena cava of a patient to prevent PEs from occurring within the patient. The embolic filter can trap embolus and prevent the embolus from travelling to the pulmonary artery.

An embolic filter can be permanent or temporary. Further, an embolic filter can be placed endovascularly, i.e., the embolic filter can be inserted into the inferior vena cava via the blood vessels of the patient. Modern filters have the capability to be compressed into relatively thin diameter catheters. Further, modern filters can be placed via the femoral vein, the jugular vein, or via the arm veins. The choice of route for installing the embolic filter can depend on the amount of blood clot, the location of the blot clot within the venous system, or a combination thereof.

The blood clot can be located using magnetic resonance imaging (MRI). Further, the filter can be placed using a filter delivery system that includes a catheter. The catheter can be guided into the IVC using fluoroscopy. Then, the filter can be pushed from the catheter and deployed into the desired location within the IVC. The filter can be made from a shape memory material that can move to an expanded configuration when exposed to body heat. However, the shape memory material may not be sufficiently stiff to maintain the filter within the IVC.

Accordingly, there is a need for an improved filter having one or more arms, legs, or a combination thereof made from a composite material.

### BRIEF DESCRIPTION OF THE DRAWINGS

FIG. 1 is a diagram of a portion of a cardiovascular system;

FIG. 2 is a plan view of a filter delivery device;

FIG. 3 is a plan view of an embolic filter in a collapsed configuration;

FIG. 4 is a plan view of the embolic filter in an expanded configuration;

FIG. 5 is a detailed view of the embolic filter; and

FIG. 6 is a cross-section view of the embolic filter taken at line 6-6 in FIG. 4.

### DETAILED DESCRIPTION OF THE DRAWINGS

An embolic filter is disclosed and includes a hub and at least one elongated member extending from the hub. The at least one elongated member includes a core and a jacket circumscribing the core. A ratio of a core diameter to a jacket diameter is at least 0.60.

In another embodiment, an embolic filter is disclosed and includes a hub. A plurality of arms can extend from the hub. Further, a plurality of legs can extend from the hub. Each of the plurality of legs is relatively longer than each of the plurality of arms. Moreover, each leg can include a core and a jacket. A ratio of a Young's modulus of the jacket to a Young's modulus of the core is at least 1.5.

In yet another embodiment, a method of making an embolic filter is disclosed and can include forming a core of an elongated member, masking a portion of the elongated member, and depositing a jacket on the core of the elongated member.

In still another embodiment, a method of making an embolic filter is disclosed and can include forming a core of an elongated member as a wire and forming a jacket of the elongated member as a tube. The method can also include stretching the core to reduce a diameter of the core and inserting the core into the jacket.

In another embodiment, a method of making an embolic filter is disclosed and can include forming a core of an elongated member as a wire and forming a jacket of the elongated member as a tube. A diameter of the wire can be slightly smaller than a diameter of the tube. Additionally, the method can include inserting the core into the jacket.

### Description of the Relevant Anatomy

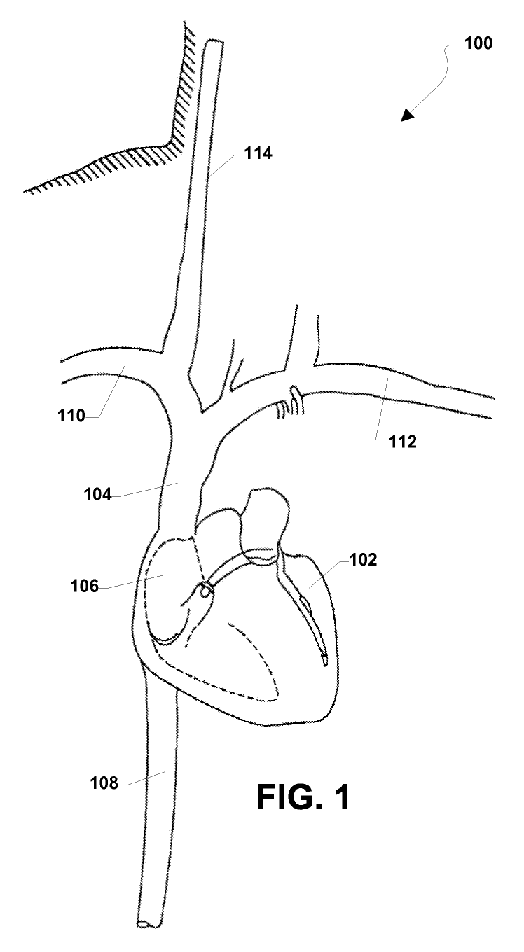

Referring to FIG. 1, a portion of a cardiovascular system is shown and is generally designated 100. As shown, the system can include a heart 102. A superior vena cava 104 can communicate with the heart 102. Specifically, the superior vena cava 104 can provide blood flow into a right atrium 106 of the heart 102 from the generally upper portion of a human body. As shown, an inferior vena cava 108 can also communicate with the heart. The inferior vena cava 108 can also provide blood flow into the right atrium 106 of the heart 102 from the lower portion of the cardiovascular system. FIG. 1 also shows a right subclavian vein 110, a left subclavian vein 112, and a jugular vein 114 that can communicate with the superior vena cava 104.

### Description of a Filter Delivery Device

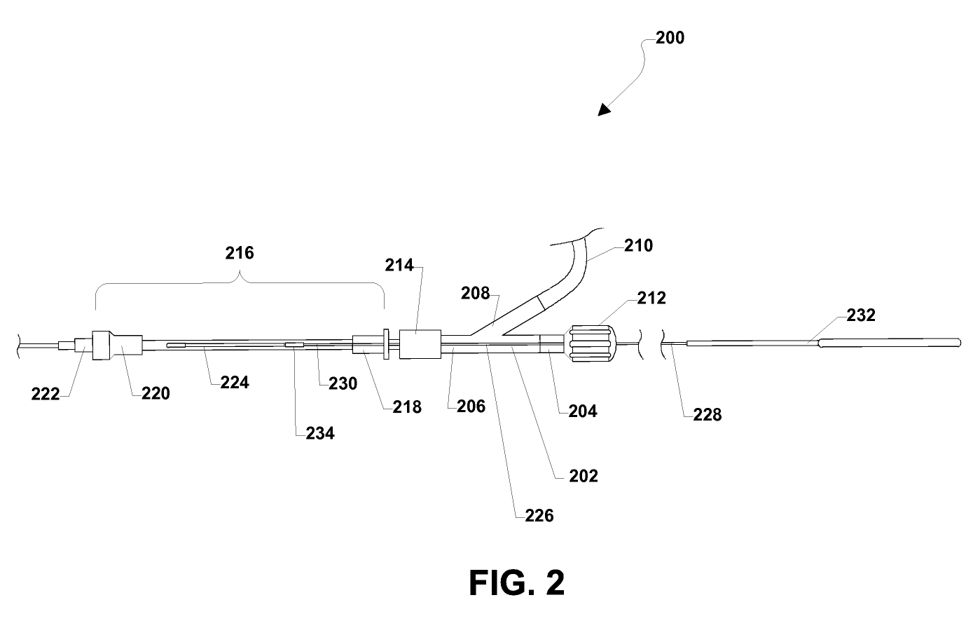

FIG. 2 illustrates a filter delivery device, designated 200. As shown, the filter delivery device can include a body 202. The body 202 of the filter delivery device 200 can be generally cylindrical and hollow. Also, the body 202 of the filter delivery device 200 can include a proximal end 204 and a distal end 206. A side port 208 can be formed in the body 202 of the filter delivery device 200 between the proximal end 204 of the body 202 and the distal end of the body 202. A saline drip infusion set 210 can be connected to the side port 208 of the body 202. In a particular embodiment, the saline drip infusion set 210 can be used to deliver saline to the patient during the delivery and deployment of an embolic filter using the filter delivery device 200.

As depicted in FIG. 2, an adapter 212 can be connected to, or integrally formed with, the proximal end 204 of the body 202 of the filter delivery device 200. Also, a filter storage tube adapter 214, or integrally formed with, can be connected to the distal end 206 of the body of the filter delivery device 200. FIG. 2 shows that the filter delivery device 200 can also include a filter storage tube 216. The filter storage tube 216 can be hollow and generally cylindrical. Further, the filter storage tube 216 can include a proximal end 218 and a distal end 220. As shown, the proximal end 218 of the filter storage tube 216 can be coupled to the filter storage tube adapter 214. An introducer catheter 222 can be connected to the distal end 220 of the filter storage tube 216.

In a particular embodiment, an embolic filter 224 can be stored within the filter storage tube 216. As shown, the embolic filter 224 can be formed into a collapsed configuration and installed within the filter storage tube 216. The embolic filter 218 can be the embolic filter described below.

FIG. 2 shows that a pusher wire 226 can be slidably disposed within the body 202 of the filter delivery device 200. The pusher wire 226 can be formed from a nickel titanium alloy, e.g., nitinol. Further, the pusher wire 226 can extend through the body 202 of the filter delivery device 200 and into the filter storage tube 216. The pusher wire 226 can include a proximal end 228 and a distal end 230. A pusher wire handle 232 can be attached to, or otherwise formed with, the proximal end 228 of the pusher wire 226. The distal end 230 of the pusher wire 226 can extend into the filter storage tube 216 attached to the body 202. Further, the distal end 230 of the pusher wire 226 can include a pusher head 234 that can contact the embolic filter 224.

During implantation of the embolic filter, the introducer catheter 222 can be threaded into the cardiovascular system of a patient, e.g., the cardiovascular system 100 described above, in order to deliver and deploy the embolic filter to the desired location with the patient. For example, the introducer catheter 222 can be threaded through the femoral vein into the inferior vena cava of the patient. A distal end of the introducer catheter 222 can include one or more radiopaque bands. Using fluoroscopy, the one or more radiopaque bands can indicate when the distal end of the introducer catheter 222 is located at or near the desired location within the inferior vena cava.

When the distal end of the introducer catheter 222 is in the desired location within the inferior vena cava, the pusher wire 226 can be moved through the body 202 of the filter delivery device 200, through the filter storage tube 216 and into the introducer catheter 222. As the pusher wire 226 is pushed through the filter storage tube 216, the embolic filter 224 is pushed from within the filter storage tube 216 into the introducer catheter 222. The embolic filter 224 can be pushed through the introducer catheter 222 until it is expelled from the distal end of the introducer catheter 222 into the inferior vena cava. Upon exiting the introducer catheter 222, the embolic filter 224 can be warmed by the body temperature of the patient. As the embolic filter 224 approaches a predetermined temperature, e.g., a normal body temperature of thirty-seven degrees Celsius (37° C.), the embolic filter 224 can move from the collapsed configuration to an expanded configuration within the inferior vena cava. In an alternative embodiment, the embolic filter 224 can move from the collapsed configuration to the expanded configuration at any temperature less than thirty-seven degrees Celsius (37° C.). Thereafter, the introducer catheter 222 can be withdrawn from the patient.

### Description of an Embolic Filter

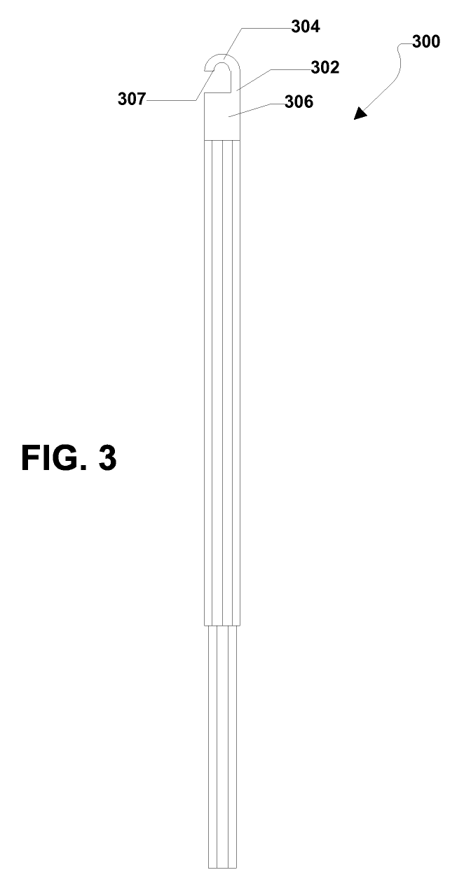

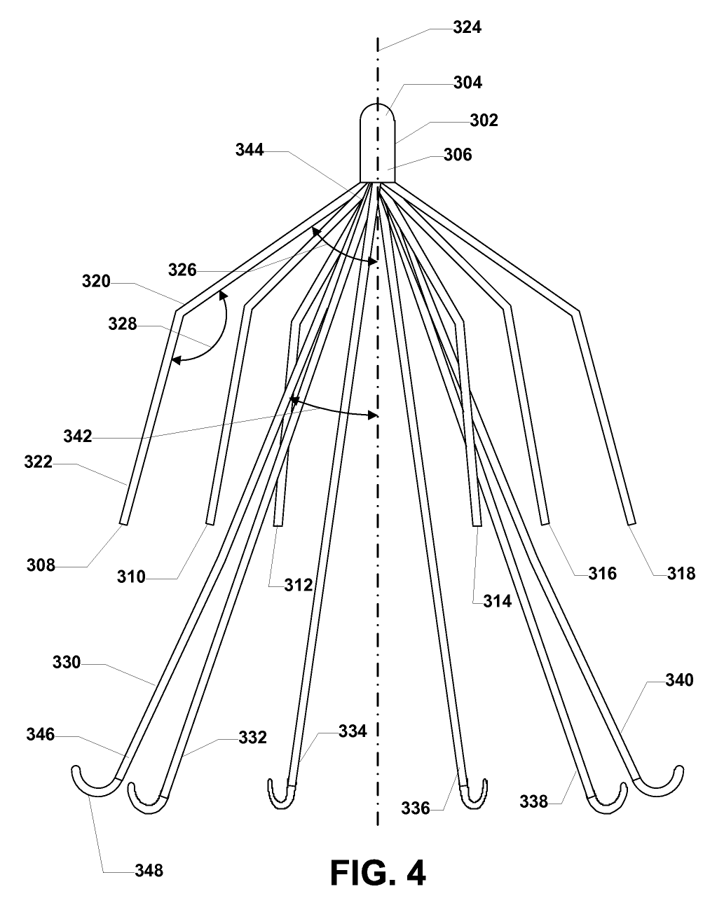

Referring now to FIG. 3 and FIG. 4, an embolic filter is shown and is generally designated 300. As depicted in FIG. 4, the embolic filter 300 can include a hub 302. The hub 302 can be generally cylindrical and hollow. Further, the hub 302 can have a proximal end 304 and a distal end 306. The proximal end 304 of the hub 302 can be closed and the distal end 306 of the hub 302 can be open. Also, the proximal end of the hub 302 can be formed with a hook 307. The hook 307 can be generally “J” shaped as shown. Alternatively, the hook 307 can be an eyehook. In a particular embodiment, the hook 307 can facilitate removal of the embolic filter 300 from within a patient. For example, a retrieval tool can be inserted into a jugular vein of a patient and moved through the jugular vein into the IVC of the patient. The retrieval tool can be engaged with the hook 307 of the embolic filter 300 and the embolic filter 300 can be withdrawn from the patient.

In a particular embodiment, several elongated members, e.g., arms, legs, or a combination thereof, can extend from the hub 302 of the embolic filter 300. For example, as indicated in FIG. 3, a first arm 308 extending from the distal end 306 of the hub 302. A second arm 310 can extend from the distal end 306 hub 302. A third arm 312 can extend from the distal end 306 of the hub 302. A fourth arm 314 can extend from the distal end 306 of the hub 302. A fifth arm 316 can extend from the distal end 306 of the hub 302. Further, a sixth arm 318 can extend from the distal end 306 of the hub 302.

Each arm 308, 310, 312, 314, 316, 318 can include a first portion 320 and a second portion 322. In the deployed, expanded configuration, shown in FIG. 3, the first portion 320 of each arm 308, 310, 312, 314, 316, 318 can extend from the hub at an angle with respect to a longitudinal axis 324 to form a primary arm angle 326.

The primary arm angle 326 can be approximately forty-five degrees (45°). In another embodiment, the primary arm angle 326 can be approximately fifty degrees (50°). In yet another embodiment, the primary arm angle 326 can be approximately fifty-five degrees (55°). In still another embodiment, the primary arm angle 326 can be approximately sixty degrees (60°). In another embodiment, the primary arm angle 326 can be approximately sixty-five degrees (65°).

The second portion 322 can be angled with respect to the first portion 320 to form a secondary arm angle 328. In particular, the second portion 322 can be angled inward with respect to the first portion 320, e.g., toward the longitudinal axis 324 of the embolic filter 300.

In a particular embodiment, the secondary arm angle 328 can be approximately twenty degrees (20°). In another embodiment, the secondary arm angle 328 can be approximately twenty-five degrees (25°). In yet another embodiment, the secondary arm angle 328 can be approximately thirty degrees (30°). In still another embodiment, the secondary arm angle 328 can be approximately thirty-five degrees (35°). In another embodiment, the secondary arm angle 328 can be approximately forty degrees (40°). In yet still another embodiment, the secondary arm angle 328 can be approximately forty-five degrees (45°).

In a particular embodiment, each arm 308, 310, 312, 314, 316, 318 is movable between a straight configuration, shown in FIG. 3, and an angled configuration, shown in FIG. 4. When the embolic filter 300 is in the pre-deployed, collapsed configuration, shown in FIG. 3, the arms 308, 310, 312, 314, 316, 318 are substantially straight and substantially parallel to the longitudinal axis 324 of the embolic filter. Alternatively, the arms 308, 310, 312, 314, 316, 318 can be at least partially twisted around the legs of the filter, described below. When the embolic filter 300 moves to the deployed, expanded configuration, shown in FIG. 4, the arms 308, 310, 312, 314, 316, 318 can move to the angled and bent configuration shown in FIG. 4.

As further illustrated in FIG. 3, the embolic filter 300 can include a first leg 330, a second leg 332, a third leg 334, a fourth leg 336, a fifth leg 338, and a sixth leg 340. Each leg 330, 332, 334, 336, 338, 340 can extend from the distal end 306 of the hub 302. In the expanded configuration, shown in FIG. 4, leg 330, 332, 334, 336, 338, 340 can extend from the hub 302 at an angle with respect to the longitudinal axis 324 to form a leg angle 342.

In a particular embodiment, the leg angle 342 can be approximately twenty degrees (20°). In another embodiment, the leg angle 342 can be approximately twenty-five degrees (25°). In yet another embodiment, the leg angle 342 can be approximately thirty degrees (30°). In still another embodiment, the primary straight leg angle 342 can be approximately thirty-five degrees (35°). In another embodiment, the leg angle 342 can be approximately forty degrees (40°). In yet still another embodiment, the leg angle 342 can be approximately forty-five degrees (45°).

In a particular embodiment, each leg 330, 332, 334, 336, 338, 340 is movable between a straight configuration, shown in FIG. 3, and an angled configuration, shown in FIG. 4. When the embolic filter 300 is in the pre-deployed, collapsed configuration, shown in FIG. 3, the legs 330, 332, 334, 336, 338, 340 are substantially straight and substantially parallel to the longitudinal axis 324 of the embolic filter. When the embolic filter 300 moves to the deployed, expanded configuration, shown in FIG. 4, the legs 330, 332, 334, 336, 338, 340 move to the angled configuration shown in FIG. 4.

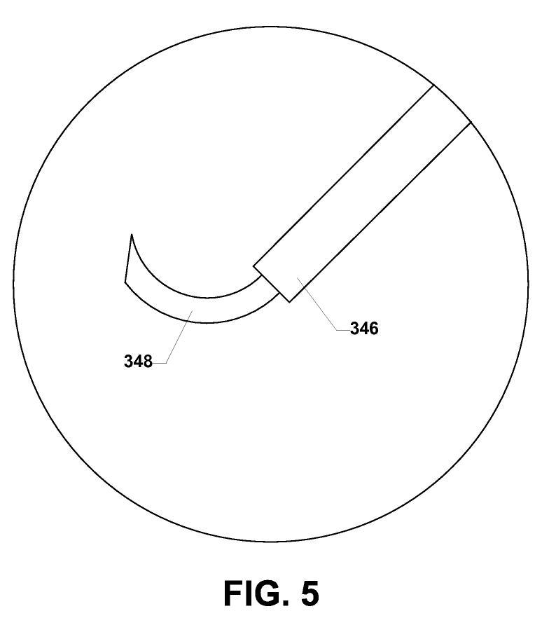

Each leg 330, 332, 334, 336, 338, 340 can include a proximal end 344 and a distal end 346. As shown in FIG. 5, the distal end 346 each leg 330, 332, 334, 336, 338, 340 can include a foot 348. Each foot 348 can be curved to form a hook or a barb. In particular each foot 348 can move from a straight configuration, shown in FIG. 3, to a curved configuration, shown in FIG. 4 and FIG. 5. As such, when the embolic filter 300 is in the collapsed configuration shown in FIG. 3, the feet 348 of the legs 330, 332, 334, 336, 338, 340 are straight. When the embolic filter 300 moves to the expanded configuration, the feet 348 are bent. Further, when the feet 348 are bent, the feet 348 can extend into and engage the inner wall of a vein in which the embolic filter is installed. The feet 348 can substantially prevent migration of the embolic filter 300. In other words, the feet 348 can engage the inner wall of the vein and substantially prevent the embolic filter 300 from moving within the vein.

In a particular embodiment, the feet 348 can substantially prevent the embolic filter 300 from migrating during normal respiratory function or in the event of a massive pulmonary embolism. Normal IVC pressures are believed to be between about two (2) and five (5) millimeters (mm) of mercury (Hg). An occluded IVC can potentially pressurize to approximately 35 mm Hg below the occlusion. The ensure stability of the embolic filter 300, the embolic filter 300 can withstand a pressure up to 50 mm Hg without migrating. When a removal pressure is applied to the filter that is greater than 50 mm Hg, the feet 348 can deform and release from the vessel wall.

The pressure required to deform the feet 348 can be converted to force using the following calculations:

Since 51.715 mm Hg=1.0 lb/in²

50 mm Hg=50/51.715=0.9668 lb/in²

For a 28 mm vena cava

A=π/4(28²)mm²=615.4 mm²=0.9539 in²

Migration force is calculated by

F=P×A

0.9668 lb/in²×0.9539 in²=0.9223 lb=418.7 g

It can be appreciated that as the diameter of the vena cava increases, the force required to resist 50 mm Hg of pressure also increases. Further, depending on the number of feet 348, the strength of each foot 348 can be calculated. For example, for an embolic filter 300 that includes six feet 348:

Foot Strength=Filter Migration Resistance Force/Number of Feet

Foot Strength=418.7/6=69.7 g

As such, each foot 348 must be capable of resisting approximately 70 grams of force in order for the embolic filter 300 to resist a 50 mm Hg pressure gradient in a 28 mm vessel. In a particular embodiment, in order to prevent excessive vessel trauma, the individual feet 348 should be relatively weak. By balancing the number of feet 348 and the individual foot strength, vessel injury can be minimized while still maintaining the ability to withstand a 50 mm Hg pressure gradient or some other predetermined pressure gradient within a range of 10 mm Hg to 120 mm Hg.

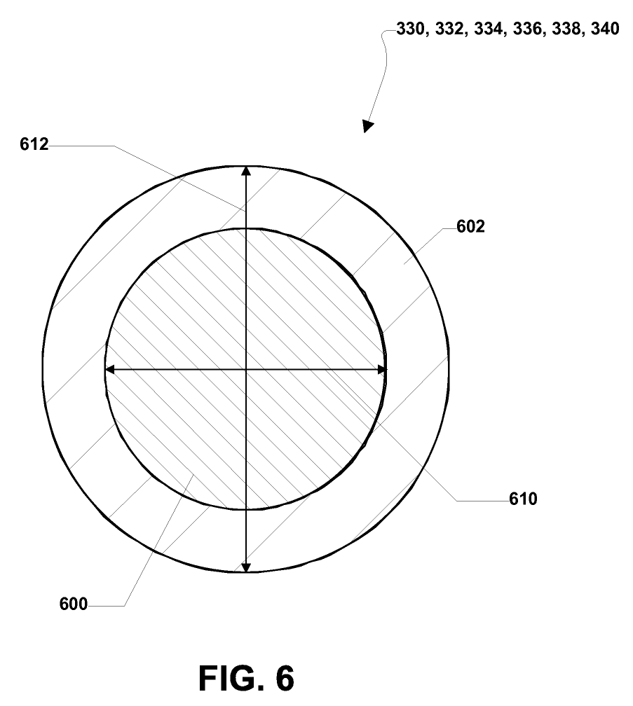

Referring now to FIG. 6, a cross-section view of a leg 330, 332, 334, 336, 338, 340 is shown. As shown, each leg 330, 332, 334, 336, 338, 340 can include a core 600 surrounded by a jacket 602. The core 600 can be relatively elastic while the jacket 602 can be relatively stiff. For example, the Young's modulus, E, of the core 600 can be less than or equal to seventy-five gigapascals (75 GPa). In another embodiment, Young's modulus, E, of the core 600 can be less than or equal to seventy gigapascals (70 GPa). In yet another embodiment, Young's modulus, E, of the core 600 can be less than or equal to sixty-five gigapascals (65 GPa). In still another embodiment, Young's modulus, E, of the core 600 can be less than or equal to sixty gigapascals (60 GPa). In yet still another embodiment, Young's modulus, E, of the core 600 can be less than or equal to fifty-five gigapascals (55 GPa). In another embodiment, Young's modulus, E, of the core 600 can be less than or equal to fifty gigapascals (50 GPa). In still yet another embodiment, Young's modulus, E, of the core 600 is not less than forty gigapascals (40 GPa).

In a particular embodiment, the core 600 can be made from a shape memory material. The shape memory material can be a shape memory polymer. Further, the shape memory material can be a shape memory metal. The shape memory metal can be a nickel titanium alloy such as nitinol.

In a particular embodiment, the Young's modulus, E, of the jacket 602 of each leg 330, 332, 334, 336, 338, 340 is greater than or equal to least seventy-five gigapascals (75 GPa). In another embodiment, the Young's modulus, E, of the jacket 602 of each leg 330, 332, 334, 336, 338, 340 is greater than or equal to one hundred gigapascals (100 GPa). In yet another embodiment, the Young's modulus, E, of the jacket 602 of each leg 330, 332, 334, 336, 338, 340 is greater than or equal to one hundred twenty-five gigapascals (125 GPa). In still another embodiment, the Young's modulus, E, of the jacket 602 of each leg 330, 332, 334, 336, 338, 340 is greater than or equal to one hundred fifty gigapascals (150 GPa). In yet still another embodiment, the Young's modulus, E, of the jacket 602 of each leg 330, 332, 334, 336, 338, 340 is greater than or equal to one hundred seventy-five gigapascals (175 GPa). In another embodiment, the Young's modulus, E, of the jacket 602 of each leg 330, 332, 334, 336, 338, 340 is greater than or equal to two hundred gigapascals (200 GPa). In still yet another embodiment, the Young's modulus, E, of the jacket 602 of each leg 330, 332, 334, 336, 338, 340 is not greater than three hundred gigapascals (300 GPa).

In a particular embodiment, the jacket 602 of each leg 330, 332, 334, 336, 338, 340 can be made from metal. For example, the metal can be titanium, tantalum, iron, or a combination thereof. Further, the iron can be an iron containing material. The iron containing material can be an iron alloy. The iron alloy can be stainless steel.

In a particular embodiment, a ratio of the Young's modulus of the jacket, E_{J}, to the Young's modulus of the core, E_{C}, is greater than or equal to one and one-half (1.5). In another embodiment, E_{J}/E_{C} is greater than or equal to two (2.0). In yet another embodiment, E_{J}/E_{C} is greater than or equal to two and one-half (2.5). In still another embodiment, E_{J}/E_{C} is greater than or equal to three (3.0). In yet still another embodiment, E_{J}/E_{C} is greater than or equal to three and one-half (3.5). In another embodiment, E_{J}/E_{C} is greater than or equal to four (4.0). In yet another embodiment, E_{J}/E_{C} is not greater than six (6.0).

In a particular embodiment, the jacket 602 is relatively shorter in length than the core 600. As such, a portion of the core 600 is exposed in order to establish the foot 348 on each leg 330, 332, 334, 336, 338, 340.

The materials of the core/jacket combination are selected in order to substantially minimize or substantially prevent corrosion of the core or the jacket. For example, a galvanic coupling current density of each leg 330, 332, 334, 336, 338, 340 is less than or equal to fifty nanoAmps per square centimeter (50 nA/cm²). In another embodiment, the galvanic coupling current density of each leg 330, 332, 334, 336, 338, 340 is less than or equal to forty nanoAmps per square centimeter (40 nA/cm²). In yet another embodiment, the galvanic coupling current density of each leg 330, 332, 334, 336, 338, 340 is less than or equal to thirty nanoAmps per square centimeter (30 nA/cm²). In still another embodiment, the galvanic coupling current density of each leg 330, 332, 334, 336, 338, 340 is less than or equal to twenty nanoAmps per square centimeter (20 nA/cm²). In another embodiment, the galvanic coupling current density of each leg 330, 332, 334, 336, 338, 340 is less than or equal to ten nanoAmps per square centimeter (10 nA/cm²). In yet still another embodiment, the galvanic coupling current density of each leg 330, 332, 334, 336, 338, 340 is not less than five nanoAmps per square centimeter (5 nA/cm²).

In a particular embodiment, a core/jacket length ratio, i.e., a ratio of a core length to a jacket length for a leg, for an arm, for a combination thereof, can be at least 0.85. In another embodiment, the core/jacket length ratio can be at least 0.86. In yet another embodiment, the core/jacket length ratio can be at least 0.87. In still another embodiment, the core/jacket length ratio can be at least 0.88. In yet another embodiment, the core/jacket length ratio can be at least 0.89. In another embodiment, the core/jacket length ratio can be at least 0.90. In yet still another embodiment, the core/jacket length ratio can be at least 0.90.

In another embodiment, the core/jacket length ratio can be at least 0.91. In still another embodiment, the core/jacket length ratio can be at least 0.92. In another embodiment, the core/jacket length ratio can be at least 0.93. In still yet another embodiment, the core/jacket length ratio can be at least 0.94. In another embodiment, the core/jacket length ratio can be at least 0.95. In another embodiment, the core/jacket length ratio can be at least 0.96. In still another embodiment, the core/jacket length ratio can be at least 0.97. In another embodiment, the core/jacket length ration is not greater than 1.0.

In order to minimize damage to the core 600, the jacket 602 is not etched away to expose the core 600. Conversely, the jacket 602 can be deposited on the core 600 along a portion of the core 600 corresponding to the core/jacket length ratio using a deposition process. For example, the deposition process can include a vapor deposition process, a powder sintering process, a vacuum deposition process, a thermal spray deposition process, or a combination thereof. The portion of the core 600 that is to remain exposed can be covered, masked, or otherwise isolated from the deposition process. Specifically, a method of making an embolic filter can include forming a core of an elongated member; masking a portion of the core, e.g., to form a foot; and depositing the jacket on the core using a deposition process. After each elongated member, e.g., each arm, each leg, or a combination thereof, is formed, the arms and legs can be inserted into the hub of the embolic filter.

Alternatively, the core 600 can be formed as a wire and the jacket 602 can be formed as a separate tube. The core 600 can be stretched in order to reduce the diameter of the core 600. Before the core 600 is stretched, the core 600 can be cooled. Once the core 600 is stretched, the core 600 can be inserted through the jacket 602. After the core 600 is inserted into the jacket 602, the resulting composite material can be heated to a predetermined temperature, e.g., to room temperature or greater, in order to return the core 600 to pre-stretched diameter. The outer diameter of the core 600 and the inner diameter of the jacket 602 can be selected so that when the core 600 is returned to the pre-stretched diameter, the core 600 can engage the jacket 602 in a press-fit tolerance, such that the core 600 cannot be easily withdrawn from the jacket 602, e.g., without mechanical aid.

The core 600 can also be formed as a wire having a slightly smaller diameter than the jacket 602. Further, the core 600 can be installed within the jacket 602 and joined to the jacket 602 using a gluing process, a welding process, or some other similar process.

As shown, the core 600 of each leg 330, 332, 334, 336, 338, 340 can have a core diameter 610. The core diameter 610 can be in a range of seven thousands of an inch to eleven thousands of an inch (0.007″−0.011″). The jacket 602 of each leg 330, 332, 334, 336, 338, 340 can have an outer jacket diameter 612. The outer jacket diameter 612 can correspond to the overall diameter of each leg 330, 332, 334, 336, 338, 340. The outer jacket diameter 612 can be in a range of thirteen thousands of an inch to twenty thousands of an inch (0.013″−0.020″).

In a particular embodiment, a ratio of the core diameter 610 to the outer jacket diameter 612 is at least 0.60. In another embodiment, the ratio of the core diameter 610 to the outer jacket diameter 612 is at least 0.65. In yet another embodiment, the ratio of the core diameter 610 to the outer jacket diameter 612 is at least 0.70. In still another embodiment, the ratio of the core diameter 610 to the outer jacket diameter 612 is at least 0.75. In yet still another embodiment, the ratio of the core diameter 610 to the outer jacket diameter 612 is at least 0.80. In another embodiment, the ratio of the core diameter 610 to the outer jacket diameter 612 is not greater than 0.85.

The core diameter 610 and the jacket diameter 612 can be chosen in order to maximize stiffness while maintaining the ability of the feet 348 to deform during filter removal. If the ratio of the core diameter 610 to the outer jacket diameter is too high, e.g., greater than 0.85, the outer jacket 602 may not be sufficiently thick enough to provide increased stiffness for the legs 330, 332, 334, 336, 338, 340 made from the composite material of the core 600 and the jacket 602.

Further, in a particular embodiment, the hub 302 of the embolic filter 300 has an outer diameter less than the inner diameter of a catheter having a French size of 7 or less. In another embodiment, the embolic filter has an outer diameter less than the inner diameter of a catheter having a French size of 6 or less. In yet another embodiment, the embolic filter has an outer diameter less than the inner diameter of a catheter having a French size of 5 or less. In each case, the outer jacket diameter 612 is substantially small enough to allow the six legs 330, 332, 334, 336, 338, 340 and the six arms 308, 310, 312, 314, 316, 318 to be fitted into the hub 302 of the embolic filter 302.

### CONCLUSION

Embodiments described herein provide a device that can be removably installed within a patient, e.g., within an inferior vena cava of a patient. The arms, the legs, or a combination thereof, can be made from a composite material.

It has been discovered that the migration to composite materials, as described herein, enable the achievement of using catheters having relatively small French sizes, e.g., less than or equal to French size of seven (7), for installation. Studies have revealed that a relatively large percentage of leg stiffness is provided by the outer skin portion of the jacket of the leg. As such, the overall diameter of each leg can be minimized while maximizing the ability to deploy the filter and maximizing the stiffness of each leg, each arm, or a combination thereof.

The use of a particular core/jacket ratio, described herein, provides notable benefits over state of the art filters, e.g., U.S. Patent Application 2005/0055045, that teach a conventional core arrangement having a ratio less than or equal to 0.56. While such arrangements have been found to be successful, embodiments described herein have discovered advantages such as minimized French size of introducer catheters.

The stiffness of the arms or the legs can be increased without increasing the overall filter-loaded profile, e.g., by simply creating an arm or a leg with a larger diameter to increase the stiffness. The increased stiffness provided by the core and jacket arrangement allows the embolic filter to remain substantially within a deployed position within a vein of a patient without migrating side-to-side or longitudinally.

Further, embodiments described herein can include a plurality of feet that are not formed using an etching process, e.g., a mechanical etching process, a chemical etching process, or a chemical etching process. Such an etching process can impart or create stress points, e.g., microscopic cracks, in the core and the core may be damaged or weakened. Over the life of a filter manufactured using an etching process, the filter can break at the areas in which the outer jacket has been removed by etching. This can result in injury to the patient due to fragments of filter material travelling through the patient's blood stream.

Since the embodiments disclosed herein are not formed using an etching process, the likelihood of one or more of the feet of the embolic filter breaking and travelling through the blood stream of the patient is substantially reduced. Further, the likelihood of filter migration due to one or more broken feet is also substantially reduced.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1. An embolic filter, comprising:
a hub; and
at least one elongated member extending from the hub, wherein the at least one elongated member comprises a core and a jacket circumscribing the core, wherein a ratio of a core diameter to a jacket diameter is at least 0.60.

2. The embolic filter of claim 1, wherein the ratio of the core diameter to the jacket diameter is at least 0.65.

3. The embolic filter of claim 2, wherein the ratio of the core diameter to the jacket diameter is at least 0.70.

4. The embolic filter of claim 3, wherein the ratio of the core diameter to the jacket diameter is at least 0.75.

5. The embolic filter of claim 4, wherein the ratio of the core diameter to the jacket diameter is at least 0.80.

6. The embolic filter of claim 5, wherein the ratio of the core diameter to the jacket diameter is not greater than 0.85.

7. (canceled)

8. (canceled)

9. The embolic filter of claim 1, wherein a Young's modulus of the core is less than or equal to 75 GPa.

10. The embolic filter of claim 9, wherein a Young's modulus of the core is less than or equal to 60 GPa.

11. The embolic filter of claim 10, wherein a Young's modulus of the core is not less than 40 GPa.

12. (canceled)

13. (canceled)

14. (canceled)

15. (canceled)

16. The embolic filter of claim 1, wherein a Young's modulus of the jacket is greater than or equal to 75 GPa.

17. The embolic filter of claim 16, wherein the Young's modulus of the jacket is greater than or equal to 75 GPa.

18. The embolic filter of claim 17, wherein the Young's modulus of the jacket is greater than or equal to 150 GPa.

19. The embolic filter of claim 18, wherein the Young's modulus of the jacket is greater than or equal to 200 GPa.

20. The embolic filter of claim 19, wherein the Young's modulus of the jacket is not greater than 300 GPa.

21. (canceled)

22. (canceled)

23. (canceled)

24. (canceled)

25. (canceled)

26. The embolic filter of claim 1, wherein a ratio of the Young's modulus of the jacket to a Young's modulus of the core is greater than or equal to 1.5.

27. (canceled)

28. (canceled)

29. (canceled)

30. (canceled)

31. The embolic filter of claim 1, wherein a galvanic coupling density of the at least one elongated member is less than or equal to 50 nA/cm².

32. The embolic filter of claim 31, wherein a galvanic coupling density of the at least one elongated member is less than or equal to 30 nA/cm².

33. The embolic filter of claim 32, wherein a galvanic coupling density of the at least one elongated member is less than or equal to 10 nA/cm².

34. (canceled)

35. An embolic filter, comprising:
a hub;
a plurality of arms extending from the hub; and
a plurality of legs extending from the hub wherein each of the plurality of legs is relatively longer than each of the plurality of arms and wherein each leg comprises a core and a jacket wherein a ratio of a Young's modulus of the jacket to a Young's modulus of the core is at least 1.5.

36. A method of making an embolic filter, comprising:
forming a core of an elongated member;
masking a portion of the elongated member; and
depositing a jacket on the core of the elongated member.

37. (canceled)

38. (canceled)

39. (canceled)

40. (canceled)

41. (canceled)

42. (canceled)

43. (canceled)

44. (canceled)

45. (canceled)

46. (canceled)
